(19) United States
(12) Patent Application Publication
(10) Pub. No.: US 2011/0184388 A1
(43) Pub. Date: Jul. 28, 2011

(54) BACTERIUM-BASED MICROROBOT FOR MEDICAL TREATMENT, OPERATION METHOD THEREOF AND TREATMENT METHOD USING THE SAME

(75) Inventors: Suk Ho Park, Gwangju (KR); Jong Oh Park, Gyeonggido (KR); Jung Joon Min, Gwangju (KR); Yeong Jin Hong, Gwangju (KR); Dong Il Cho, Seoul (KR); Jong Mo Seo, Seoul (KR); Jennifer Hyunjong Shin, Seoul (KR); Heung Soo Shin, Seoul (KR); Jung Yul Park, Seoul (KR)

(73) Assignee: INDUSTRY FOUNDATION OF CHONNAM NATIONAL UNIVERSITY, Gwangju (KR)

(21) Appl. No.: 13/058,656

(22) PCT Filed: Dec. 30, 2008
(86) PCT No.: PCT/KR2008/007769

§ 371 Date: Feb. 11, 2011

(30) Foreign Application Priority Data
Oct. 31, 2008 (KR) 10-2008-0108011

(51) Int. Cl. A61M 37/00

(52) U.S. Cl. 604/891.1

(57) ABSTRACT

Provided are a bacterium-based microrobot for medical treatment, an operation method thereof, and a treatment method using the same. The bacterium-based microrobot can be propelled by the flagellum movement of bacteria, can be directed toward a target lesion by the ability of bacteria to recognize the lesion, can be monitored for how many the microrobot targets the lesion, and can directly or indirectly treat the lesion by the proliferation of bacteria through self-division in the lesion. The bacteria may be genetically manipulated to be resistant to immune responses and produce a material inhibitory of the growth of affected cells.

### TECHNICAL FIELD

The present invention relates to a bacterium-based microrobot for medical use, a method of operating the same, and a treatment method using the same. More particularly, the present invention relates to a bacterium-based microrobot taking advantage of various bacterial properties including motility, recognition, fluorescence, and healing effects. The bacteria may be mutated through genetic manipulation to exhibit these properties, thereby allowing the microrobot to be useful in the treatment of lesions.

### BACKGROUND ART

There have been suggestions made for using medical microrobots for the examination and therapy of the digestive tract such as those shown in FIG. 1 and for intravascular therapy such as that shown in FIG. 2.

Generally, a microrobot 110 comprises various components including a location information providing unit 120, a driving unit 130, a treatment unit 140, a robot control unit 150, a data transmission/reception unit 160, radio power reception unit 170, a sensing unit 180, and a power unit 190.

Among the components, the driving unit 130, the sensing unit 180, and the power unit 190 are the most important.

Severe limitations are imparted to the sizes of the driving unit 130, the sensing unit 180 and the power unit 190 due to the inherent properties of microrobots.

For the most part, for example, the driving unit 130 employs a micromotor which is typically 1˜2 mm in size.

In addition, intelligent materials, such as shape memory alloys and EAP (Electro-Active Polymer), are applied for the construction of micromotors, but with limitations.

For the power unit 190, a battery is typically used. However, a microrobot is difficult to equip with a battery of sufficient capacity due to the size limitation thereof.

In order to solve these problems, studies have been conducted on the use of biological cells in microrobots, particularly the use of cardiomyocytes that spontaneously contract. In this case, cardiomyocytes are cultured in a microstructure to give a contractile force. Advantageously, the contractile force of cardiomyocytes may be used as a driving force for various functions.

However, it is difficult to control cardiomyocyte-actuated microsystems and to establish conditions suitable for retaining the contractile force of cardiomyocytes.

In particular, when the cardiomyocyte-actuated microsystems are applied to the body, they suffer from the disadvantage of inducing an immune response.

### DISCLOSURE

### Technical Problem

Accordingly, the present invention has been made keeping in mind the above problems occurring in the prior art, and an object of the present invention is to provide a bacterium-based microrobot for medical treatment which can be propelled by the flagellum movement of bacteria, can be directed toward a target lesion by the ability of bacteria to recognize the lesion, can be monitored for how many of the microrobots target the lesion, and can directly or indirectly treat the lesion by the proliferation of bacteria through self-division in the lesion.

### Technical Solution

In accordance with the present invention, the object can be accomplished by a provision of a bacterium-based microrobot (400) for medical inspection and treatment, comprising:

a capsule-type microstructure designed to carry and discharge a drug; and bacteria, attached to an outer circumference and rear side of the microstructure, having a self-propelling flagellum or flagella allowing movement in all directions, which recognize affected environments or cells to guide the microrobot to a lesion,

the microstructure comprising:

a sampling port, provided to an outer wall of the microstructure, for taking a blood sample thereinto;

a reagent unit for storing and effusing a reagent to test a reaction with a target lesion in advance, when a blood sample is taken and introduced into the microstructure;

a mixing unit for mixing the blood sample from the sampling port with the reagent from the reagent unit;

a diagnostic unit for analyzing results of a reaction between the blood sample and the reagent;

a control unit for determining a treatment to be administered according to results of the analysis of the diagnostic unit;

a drug unit for effusing a drug according to an instruction of the control unit; and

a discharging unit for delivering the drug effused by the drug unit to the lesion with the aid of a pump.

In the microrobot, the bacteria have flagella for self-propulsion and are attached to the outer circumference of the microstructure. Thus, the microrobot can be directed toward a lesion as the bacteria move toward and search for a lesion due to the motility and cognition thereof. Also, the microrobot, when reaching a target lesion, discharges a drug onto the lesion and allows the bacteria to proliferate in the lesion, thereby treating the disease.

Also, a bacterium-based microrobot is provided for treating a disease by discharging a drug onto a lesion in response to a diagnostic analysis of the lesion and proliferating bacteria within the lesion to remove the lesion.

A treatment method using the bacterium-based microrobot for medical inspection and treatment is provided, comprising:

propelling the microrobot toward a lesion by use of bacteria having motility and cognition;

taking a blood sample through a sampling port when the microrobot is positioned at the lesion by the bacteria;

effusing a reagent for reacting with the lesion from a reagent unit when the blood sample is introduced through the sampling port into the microrobot;

sucking and mixing the blood sample from the sampling port and the reagent from the reagent unit in a mixing unit;

transferring the mixture of the blood and the reagent from the mixing unit to a diagnostic unit in which the mixture is analyzed.

Determining a treatment manner in a control unit in response to the analysis result of the diagnostic unit and sending a control signal;

discharging a drug from a drug unit in accordance with the signal of the control unit; and

spraying the drug over the external lesion through a discharging unit with the aid of a pump.

### Advantageous Effects

Furthermore, the bacteria used in the microrobot of the present invention are suitably maintained on the microstructure and may be genetically manipulated to recognize and move toward a target lesion and to perform various therapeutic functions (drug delivery, siRNA, and proliferation).

Based on the genetically manipulated bacteria 200 which also act as an actuator and a sensor thanks to the motility and cognizance thereof, the microrobot can be miniaturized.

In addition, the bacterium-based microrobot of the present invention can directly reach a target lesion thanks to the motility and recognition of the bacteria and thus may locally treat the lesion at higher efficiency than can a conventional treatment. Further, the microrobot in accordance with the present invention is designed to treat a target lesion externally and internally through drug delivery, growth inhibition with siRNA and bacterial healing effects. Accordingly, the microrobot of the present invention is expected to suggest novel and effective target therapy methods.

### DESCRIPTION OF DRAWINGS

FIG. 1 is of photographs showing conventional microrobots for medical inspection in the digestive tract.

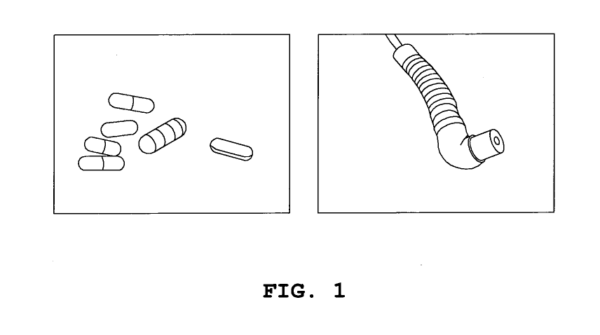

FIG. 2 is schematic diagram showing a conventional microrobot for use in intra vascular treatment.

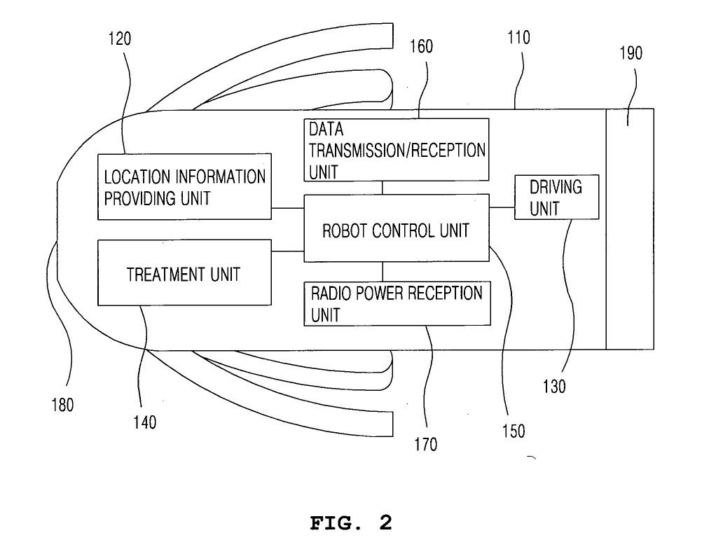

FIG. 3 is a view showing a bacterium-based microrobot system for medical treatment in accordance with the present invention.

FIG. 4 is a schematic diagram showing a bacterium-based microrobot for medical treatment in accordance with the present invention.

FIG. 5 is a block diagram showing a method of operating the bacterium-based microrobot for medical treatment in accordance with the present invention.

FIG. 6 is a schematic diagram showing a microstructure for inhibiting cancer growth through the use of siRNA synthesis bacteria, useful in the bacterium-based microrobot for medical treatment in accordance with an embodiment of the present invention.

FIG. 7 is a schematic diagram showing a microstructure for delivering a drug by use of drug-activating enzyme synthesis bacteria, useful in the bacterium-based microrobot for medical treatment in accordance with another embodiment of the present invention.

FIG. 8 is a schematic diagram showing a microstructure employing siRNA and enzyme synthesis bacteria, useful in the bacterium-based microrobot for medical treatment in accordance with a further embodiment of the present invention.

<Descriptions for Main Numerals in Drawing>

| | |
|---|---|
| 200: Bacteria | 202: Flagella |
| 210: siRNA bacteria | 220: Enzyme synthesis bacteria |
| 300: Microstructure | 310: Sampling port |
| 320: Reagent unit | 330: Mixing unit |
| 340: Diagnostic unit | 350: Control unit |
| 360: Drug unit | 362: Drug |
| 370: Pump | 380: Discharging port |
| 392: Chamber | 394: Chamber 1 |
| 396: Chamber 2 | 398: Microvalve |
| 400: Microrobot | |

### BEST MODE

In accordance with an aspect thereof, the present invention provides

a bacterium-based microrobot for medical inspection and treatment, comprising: a capsule-type microstructure designed to carry and discharge a drug; and bacteria, attached to an outer circumference and rear side of the microstructure, having a self-propelling flagellum or flagella allowing movement in all directions, which recognize affected environments or cells to guide the microrobot to a lesion, whereby the microrobot can deliver the drug selectively to the lesion and allow the bacteria to proliferate within the lesion, so as to treat the lesion.

In the bacterium-based microrobot, the bacteria, attached to the outer circumference and rear side of the capsule-type microstructure, has motility towards and recognition for a lesion, and the capsule-type microstructure comprises:

a sampling port, provided to an outer wall of the microstructure, for taking a blood sample thereinto;

a reagent unit for storing and effusing a reagent to test a reaction with a target lesion in advance, when a blood sample is taken and introduced into the microstructure;

a mixing unit for mixing the blood sample from the sampling port with the reagent from the reagent unit;

a diagnostic unit for analyzing results of a reaction between the blood sample and the reagent;

a control unit for determining a treatment to be administered according to the results of the analysis of the diagnostic unit;

a drug unit for effusing a drug according to an instruction of the control unit; and

a discharging unit for delivering the drug effused by the drug unit to the lesion with the aid of a pump.

In accordance with another aspect thereof, the present invention provides a bacterium-based microrobot for medical treatment, designed to treat a target lesion externally by discharging a drug over the lesion and internally by allowing the bacteria to proliferate in the lesion.

Hereinafter, embodiments of the present invention will be described in detail with reference to the attached drawings.

Hereinafter, embodiments of the present invention will be described in detail with reference to the attached drawings.

Prior to describing our invention as related to the embodiment shown in the accompanying drawing, it is our intention that the invention be not limited by any of the details of description, unless otherwise specified, but rather be construed broadly within its spirit and scope as set out in the accompanying claims.

While there has been described what is at present considered to be the preferred embodiment of the invention, it will be understood that various modifications may be made therein, and it is intended to cover in the appended claims all such modifications as fall within the true spirit and scope of the invention.

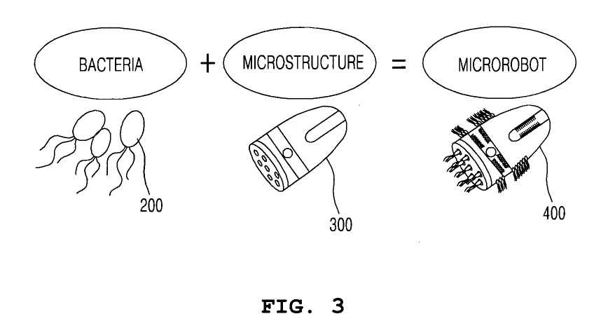

With reference to FIG. 3, a bacterium-based microrobot system for medical treatment in accordance with the present invention is shown.

As seen in this drawing, the microrobot 400 according to the present invention takes advantage of the properties of bacteria 200, including motility, cognizance, fluorescence, healing effects, etc.

First, the bacteria 200 may use flagella 202 for self-propulsion. Also, the bacteria 200 are able to recognize affected environments or cells. Next, the bacteria 200 may express fluorescence. Further, the bacteria 200 may be curative of diseases (e.g. some types of cancer). In addition, the bacteria 200 may be insensitive to the immune response of the body. Moreover, the bacteria 200 increase their population size through self-division. Finally, the bacteria 200 can be imparted with the above-mentioned properties through genetic manipulation.

Utilizing such properties of the bacteria 200, the microrobot 400 for medical use in accordance with the present invention is accordingly based on the bacteria 200.

That is, the microrobot 400 according to the present invention is constructed on the basis of bacteria 200 which have flagella allowing movement in all directions, which recognize affected environments or cells and approach lesions, in which quantitatively detectable fluorescence is expressed, which are insensitive to the immune response of the body, proliferate by self-division, and are directly or indirectly curative of diseases.

In the microrobot 400 of the present invention, bacteria 200 capable of being propelled by the flagella thereof are attached, along the outer circumference, to a microstructure in a capsule form. Thus, the microrobot 400 takes advantage of the motility and cognizance of the bacteria 200 so as to search for and move toward lesions, and treats the disease with the bacteria 200 themselves and by discharging chemicals 362 to the target lesions.

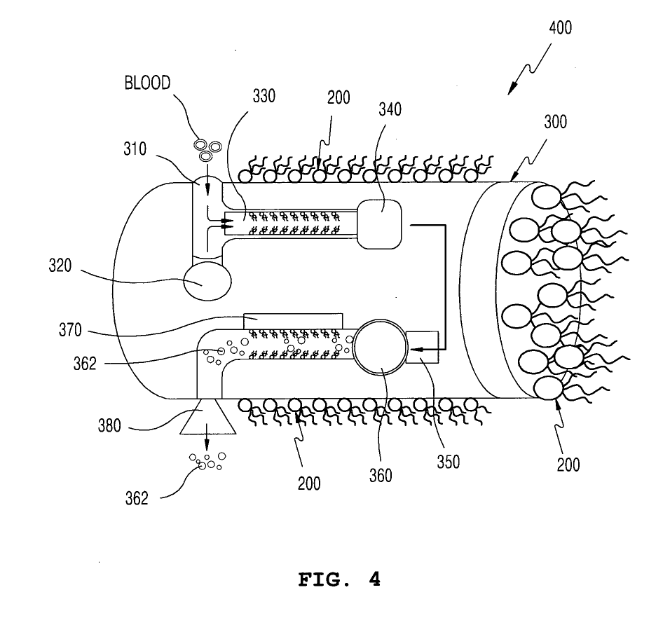

Referring to FIG. 4, a bacterium-based microrobot for medical treatment in accordance with the present invention is shown.

As seen in this figure, the microrobot 400 for use in medical inspection and treatment comprises a microstructure 300 in the form of a capsule with bacteria 200 attached to the rear side and circumference thereof.

The bacteria 200 attached to the outer walls of the microrobot 400 have mobility and cognition of lesions.

In the microrobot 400 with bacteria 200 attached to the circumference thereof, a sampling port 310 for taking a blood sample is provided to an outer wall of the microstructure 300. When a blood sample is taken and introduced into the microstructure, a reagent unit 320 located adjacent to the sampling port 310 effuses a reagent to test a reaction with a target lesion in advance.

Also, the microrobot 400 is structured to comprise a mixing unit 330 for mixing the blood sample from the sampling port 310 with the reagent from the reagent unit 320, a diagnostic unit 340 for analyzing the results of the reaction between the blood sample and the reagent, a control unit 350 for determining a treatment to be administered according to the results of the analysis of the diagnostic unit 340, and a drug unit 360 for effusing a drug 362 according to the instruction of the control unit 350. A discharging unit 380 is provided for transferring the drug 362 effused by the drug unit 360 to lesions with the aid of a pump 370.

In this structure, the microrobot 400 can be propelled and directed toward a target lesion as the bacteria attached to the circumference and rear side of the microstructure, which is in a capsule form, are propelled using flagellum movement and are cognizant of the environment and the target lesion. Once the target lesion is reached, the microrobot 400 treats the disease by discharging a drug 362 thereto and by utilizing the therapeutic action of the bacteria 200.

The motility of bacteria 200 is attributed to the movement of a specialized flagellum 202, the rotation of which causes the entire bacterium to move forward in a corkscrew-like motion. This flagellum is approximately 10,000 nanometers in length with a diameter of approximately 20 nanometers.

For the mechanical movement of the flagella 202, the bacteria 200 utilize the chemical energy of the medium.

This motility of bacteria 200 is used as a driving force for the bacterium-based microrobot 400. In principle, the bacteria for use in this microrobot must be selected as being of high motility. Genetic manipulation may result in a bacterial species with high motility.

Further, the bacteria 200 attached to the microstructure of the microrobot are genetically manipulated to be resistant to immune responses. Accordingly, the genetically manipulated bacteria 200 can safely move the microrobot 400 to the target lesion in spite of being subjected to cellular attacks.

In the body, bacteria are typically sensitive to and killed by macrophages which induce immune responses. Thus, the bacteria 200 for use in the present invention must be genetically manipulated to be insensitive to the immune responses in order to guide the microstructure to the target lesion.

Showing these properties, the bacteria 200 attached to the outer circumference and rear side of the microstructure can propel the microrobot 400 despite any cellular attack.

Further, the ability of the bacteria to recognize an affected environment or cell is utilized by the microrobot 400 of the present invention.

Moreover, the bacteria 200 may show chemotaxis, phototaxis, magnetotaxis and/or aerotaxis (anaerobic). These taxes play a critical role in a bacterium's cognition of target lesions and can be used to regulate the motility of the bacterium 200.

Particularly, chemotaxis not only allows the bacteria 200 to search for lesions, but also can be applied to the transportation of the drug 362 and thus the treatment of the disease by the microrobot 400.

In addition, thanks to the fluorescence expressed by the bacteria 200, the microrobot 400 can be monitored in order to determine its location within the body and to determine whether it has reached the target lesion.

Thus, the bacteria 200 for use in the present invention must express a fluorescent protein. This expression may be achieved by genetic manipulation.

The fluorescence expressed by the bacteria 200 makes it possible to detect the position of the microrobot 400 in the body, thereby allowing tracing of the moving path of the bacteria and determining the location of the lesion.

When reaching the lesion, the bacteria attached to the microrobot 400 can increase in number geometrically through self-division and thus attack the lesion to heal the disease.

That is, although a very small number of the bacteria 200 reach the lesion, their population increases by geometric progression to a number sufficiently large to treat the disease.

Also, because the bacteria 200 proliferate by self-division, they may be readily maintained in a quantity necessary for constructing the microrobots 400.

After being moved to the lesion by the bacteria, the microrobot treats the disease by spraying the drug 362 onto the lesion according to the analysis of the diagnostic unit 340 while the bacteria 200 enter and proliferate in the lesion to effect an internal treatment.

Preferably, the bacteria 200 are those that can treat a specific disease (e.g., cancer). Thus, the bacteria 200 are chemotactic for chemicals expressed specifically by cancer cell populations.

In most cases, bacteria are killed by the attack of macrophages. However, the internal space of cancer cells is known to be a safety zone for bacteria. Thus, the bacteria 200 can proliferate safely within the internal space of cancer cells. As the population of the bacteria 200 increases, the cancer cell population decreases.

The above-mentioned properties are inherent to some bacteria or may be imparted to the bacteria 200 by genetic manipulation. Further, genetic engineering technology allows these properties to be activated or inactivated.

Since these properties can be implemented to one bacterium 200 by current art genetic manipulation, the bacteria 200 which are genetically engineered in a preferable manner can be used in the construction of the microrobot 400 according to the present invention.

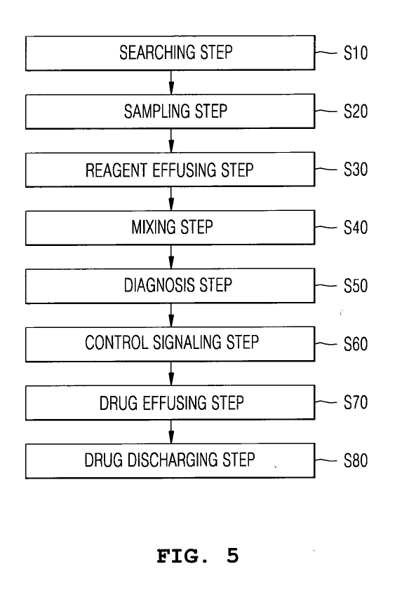

Turning to FIG. 5, illustrated in a stepwise manner is how the microrobot 400 for medical inspection and treatment in accordance with the present invention operates.

First, the microrobot 400 is propelled toward a lesion by the bacteria 200 which have motility and cognition (S10).

When positioned at the lesion by the bacteria 200, the microrobot 400 takes a blood sample through the sampling port 310 (S20).

The blood sample is introduced through the sampling port 310 into the microrobot 400 and the reagent unit 320 effuses a reagent for reacting with the lesion (S30).

Then, the blood sample from the sampling port 310 and the reagent from the reagent unit 320 are sucked and mixed in the mixing unit 330 (S40). Next, the resulting mixture of the blood and the reagent is transferred from the mixing unit 330 to the diagnostic unit 340 in which it is analyzed (S50).

Afterwards, the control unit 350 determines a treatment manner in response to the analysis result of the diagnostic unit 340 and sends a control signal (S60).

The drug unit discharges the drug 362 in accordance with the signal of the control unit 350 and the drug 362 is sprayed over the external lesion through the discharging unit 380 with the aid of the pump 370 (S80).

The microrobot 400 takes advantage of various properties endowed to the bacteria 400 in effecting therapeutic functions on lesions.

As described above, the microrobot 400 in accordance with the present invention can be actuated by bacteria 200 which have motility and cognition of target lesions and thus can its movement be controlled using the bacteria 200. The microrobot 400 takes a blood sample from an environment around the target lesion with the aid of the pump 370 and mixes the sample with a reagent in the mixing unit 330 to identify the target lesion. After the reaction between the sample and the reagent is analyzed in the microfluidic structure, a signal is generated by the control unit and sent to the drug unit to discharge the drug 362 to the lesion with the aid of the pump 370.

In addition, the bacteria 200 go through the lesion and proliferate therein to treat the lesion.

In an embodiment of the present invention, the bacterium-based microrobot 400 is propelled to a target lesion by the bacteria 200 attached thereto and the bacteria are proliferated within the lesion to perform medical treatment for the lesion.

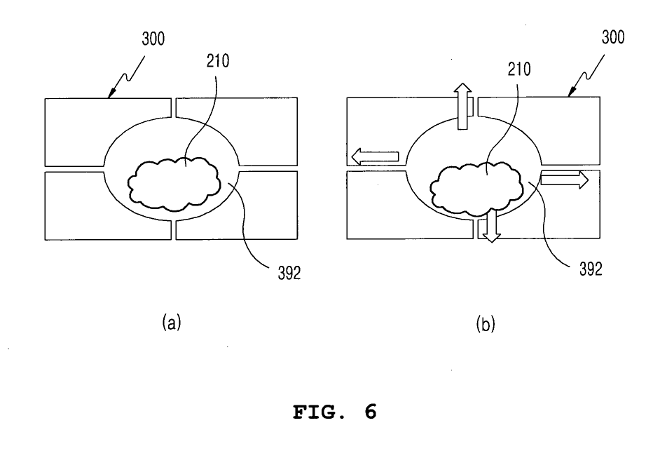

Referring to FIG. 6, a method of treating cancer using the microrobot 400 with genetically manipulated siRNA bacteria 210 confined therein in accordance with an embodiment of the present invention is illustrated.

For this, a gene which can be transcribed to siRNA is inserted into the bacteria 200 to produce siRNA bacteria 210.

A microstructure 300 is structured to have a chamber 392 within which the siRNA bacteria 201 are protected and from which the siRNA bacteria 210 are secreted through a channel to the outside. Accordingly, the siRNA bacteria 210 are not blocked from attack by macrophages.

A microrobot 400 in accordance with an aspect of the present invention may be constructed by attaching the bacteria 200 to the outer circumference and rear side of the microstructure 300 which comprises a chamber with genetically manipulated siRNA bacteria 210 confined therein. When the microrobot reaches a target lesion by the self-propelling action of the bacteria 200, the siRNA bacteria 210 are discharged and function to remove the lesion.

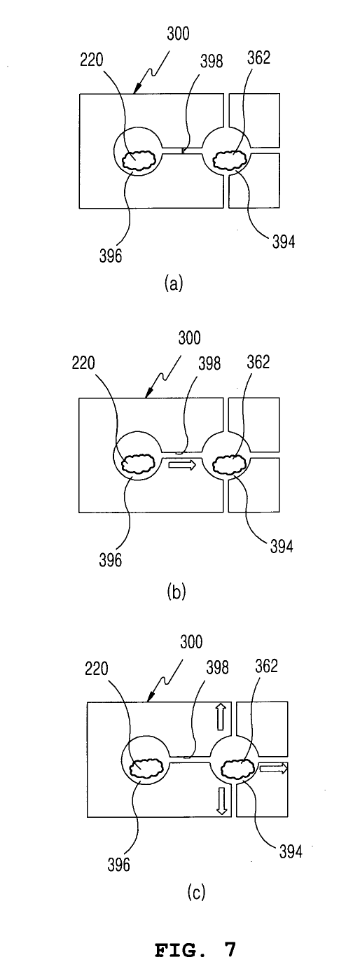

With reference to FIG. 7, a method of treating a lesion by using the microrobot 400 with enzyme synthesis bacteria 220 provided therefore in accordance with another embodiment of the present invention is illustrated. This method utilizes a chemical as a direct therapeutic agent for a target lesion.

Generally, anti-cancer agents show significant side effects upon systemic administration.

In a therapeutic strategy, an anti-cancer agent is chemically inactivated by being linked to a molecule and the resulting inactive anti-cancer agent is moved around cancer cells, followed by breaking the link to activate the anti-cancer agent. Thus, the toxic anti-cancer agent is selectively transferred at a high concentration only to the cancer cells.

According to this strategy, as seen in FIG. 7, chemically inactivating molecules are immobilized onto the wall of a first chamber 394 and linked to a drug. Enzyme synthesis bacteria 220 which can express an enzyme able to activate the drug are cultured. These cultured enzyme synthesis bacteria 220 are placed in a second chamber 396.

To the outer wall of the microstructure 300 structured in this manner, bacteria 200 are attached. When a target lesion has been reached, the microstructure 300 opens a microvalve 398.

The enzyme synthesis bacteria 220 move from the second chamber 396 to the first chamber 394 in which the enzyme secreted from the bacteria 220 breaks the link between the drug and the chemically inactivating molecules, thus converting the inactive pre-drug to the active drug 362. Finally, the drug 362 is discharged collectively to the lesion from the microstructure 300. Herein, the microstructure 300 functions to protect the enzyme synthesis bacteria 220 from phagocytosis and as a nest for the bacteria 200 and the drug 362 and as a channel for transferring the enzyme and the drug.

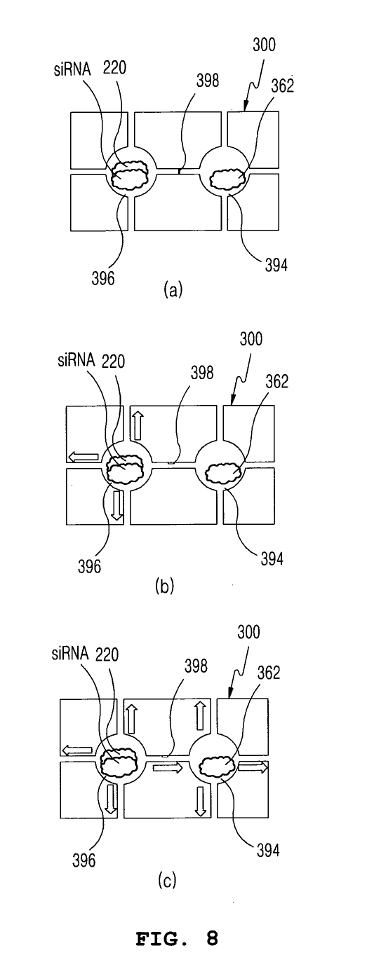

With reference to FIG. 8, a method of treating a lesion through the use of siRNA and enzyme synthesis bacteria 220 in combination in accordance with a further embodiment of the present invention is illustrated. For this, bacteria are genetically manipulated to produce siRNA and an activating enzyme, simultaneously.

While the enzyme secreted from the bacteria is used to activate the drug 362, the siRNA is utilized to suppress the growth of cancer cells. The bacteria are attached to the outer walls of the microstructure 300 thus fabricated. After being moved to a target lesion by the bacteria 200, the microrobot 400 is operated to open the microvalve 398 in the vicinity of the lesion. This method features the medical treatment using the three means of cancer suppression by siRNA, chemical therapy with the drug 362, and treatment through bacterial proliferation within the lesion.

In this embodiment, the microrobot 400 comprises a microstructure with the bacteria 200 attached to an outer circumference and rear side thereof and is moved to a target lesion by the flagellum movement of the bacteria 200. The microstructure is designed to have a first chamber 394 and a second chamber 396 which serve as reservoirs for a chemically inactivated drug 362 and siRNA and enzyme synthesis bacteria 220 respectively and to open a microvalve 398 in the vicinity of the target lesion to move the siRNA and enzyme synthesis bacteria 220 from the second chamber 396 to the first chamber 394 in which the enzyme secreted from the bacteria breaks a link between the drug and the chemically inactivating molecule. Then, the drug 362 thus activated is discharged to the outside of the microstructure 300 to treat the lesion.

As described above, the microrobot 400 of the present invention may have two or more therapeutic means including the drug 362 toxic to the affected cells, and the bacteria 200 attached thereto.

Also, the microrobot 400 is propelled toward a target lesion by taking advantage of the motility and cognizance of the bacteria 200 and transfers the drug 362 selectively to the lesion so as to effectively treat the disease after reaching the lesion.

Thanks to the fluorescence of the bacteria, the microrobot can be monitored for the position thereof within the body and for whether it has accurately reached the target lesion.

In combination with the drug 362 sprayed over the lesion from the microrobot 400, the bacteria 200 plays a therapeutic role in the treatment of the disease.

That is, the bacteria enter the lesion and proliferate therein to remove the lesion.

Hence, the drug 362 attacks the lesion from the outside while the bacteria 200 conduct a therapeutic function inside the lesion.

The bacterium-based microrobot 400 for medical treatment in accordance with the present invention takes advantage of bacterial cognition for affected environments and cells in targeting and moving toward lesions, and of bacterial fluorescence expression in being analyzed for how many of the microrobots target the lesion, and of the insensitivity to immune responses and the proliferation by self-division of the bacteria 200 in treating the lesion directly and indirectly.

Furthermore, the bacteria 200 used in the microrobot 400 of the present invention are suitably maintained on the microstructure 300 and may be genetically manipulated to recognize and move toward a target lesion and to perform various therapeutic functions (drug delivery, siRNA, and proliferation).

Based on the genetically manipulated bacteria 200 which also act as an actuator and a sensor thanks to the motility and cognizance thereof, the microrobot 400 can be miniaturized.

The foregoing invention has been described in terms of preferred embodiments. However, those skilled, in the art will recognize that many variations of such embodiments exist. Such variations are intended to be within the scope of the present invention and the appended claims.

We claim:

1. A bacterium-based microrobot for medical inspection and treatment, comprising:
a capsule-type microstructure designed to carry and discharge a drug; and
bacteria, attached to an outer circumference and rear side of the microstructure, having a self-propelling flagellum or flagella allowing movement in all directions, which recognize affected environments or cells to guide the microrobot to a lesion,
whereby the microrobot can deliver the drug selectively to the lesion and allow the bacteria to proliferate within the lesion, so as to treat the lesion.

2. The bacterium-based microrobot according to claim 1, wherein the bacteria, attached to the outer circumference and rear side of the capsule-type microstructure have motility and recognition for a lesion, and the capsule-type microstructure comprises:
a sampling port, provided on an outer wall of the microstructure, for taking a blood sample thereinto;
a reagent unit for storing and effusing a reagent to test a reaction with the lesion in advance, when a blood sample is taken and introduced into the microstructure;
a mixing unit for mixing the blood sample from the sampling port with the reagent from the reagent unit;
a diagnostic unit for analyzing results of a reaction between the blood sample and the reagent;
a control unit for determining a treatment to be administered according to results of the analysis of the diagnostic unit; and
a drug unit for effusing a drug according to an instruction of the control unit; and
a discharging unit for delivering the drug effused by the drug unit to the lesion by using a pump.

3. The bacterium-based microrobot according to claim 2, wherein the bacteria attached to the outer circumference and rear side of the microstructure are genetically manipulated to be resistant to immune responses, thereby ensuring that the microrobot safely moves to the target lesion in spite of cellular attacks.

4. The bacterium-based microrobot according to claim 2, wherein the bacteria express a fluorescent protein which can be used to detect a position of the microrobot in a body and to monitor whether the microrobot has reached the lesion.

5. The bacterium-based microrobot according to claim 2, wherein the bacteria attached to the outer circumference and rear side of the microstructure, after reaching a lesion, increase in number geometrically through self-division and thus attack the lesion to heal a disease.

6. The bacterium-based microrobot according to claim 2, wherein the microrobot guided to the lesion by the bacteria sprays the drug onto the lesion according to an analysis of the diagnostic unit and the bacteria enter and proliferate in the lesion to effect an internal treatment.

7. A method of operating a microrobot for medical inspection and treatment, comprising:
propelling the microrobot toward a lesion by using bacteria having motility and cognition;
taking a blood sample through a sampling port when the microrobot is positioned at the lesion by the bacteria;
effusing a reagent for reacting with the lesion from a reagent unit when the blood sample is introduced through the sampling port into the microrobot;
sucking and mixing the blood sample from the sampling port and the reagent from the reagent unit into a mixing unit to form a mixture;
transferring the mixture of the blood and the reagent from the mixing unit to a diagnostic unit in which the mixture is analyzed;
determining a treatment manner in a control unit in response to an analysis result of the diagnostic unit and sending a control signal;
discharging a drug from a drug unit in accordance with the control signal of the control unit; and
spraying the drug over the external lesion through a discharging unit by using a pump.

8. A method of treating a disease with a bacterium-based microrobot for medical inspection and treatment, comprising:
moving the microrobot to a lesion by using bacteria which are attached to a microstructure of the microrobot and which have been genetically manipulated;
proliferating the bacteria in the lesion to effect internal treatment.

9. The method according to claim 8, wherein the microstructure comprises a first internal chamber storing siRNA bacteria, the first internal chamber externally discharging siRNA produced by the siRNA bacteria in vicinity of the lesion to effect a therapeutic function.

10. The method according to claim 8, wherein the microstructure comprises a first internal chamber for storing a chemically inactivated drug and a second internal chamber for storing enzyme synthesis bacteria and is operated to open a microvalve in a vicinity of the lesion to move the enzyme synthesis bacteria from the second chamber to the first chamber in which the enzyme secreted from the bacteria activates the chemically inactivated drug which is in turn discharged onto the lesion.

11. The method according to claim 8, wherein the microstructure comprises a first internal chamber for storing a chemically inactivated drug and a second internal chamber for storing siRNA and enzyme synthesis bacteria and is operated to open a microvalve in a vicinity of the lesion to move the enzyme synthesis bacteria from the second chamber to the first chamber in which the enzyme secreted from the bacteria activates the chemically inactivated drug which is in turn discharged onto the lesion while the siRNA produced by the bacteria interfere with a growth of affected cells of the lesion.
